(19) United States
(12) Patent Application Publication
(10) Pub. No.: US 2008/0033029 A1
(43) Pub. Date: Feb. 7, 2008

(54) Treating Digestive Tract Conditions

(75) Inventor: Gianrico Farrugia, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 11/568,502

(22) PCT Filed: May 6, 2005
(86) PCT No.: PCT/US05/15830

§ 371 Date: Aug. 13, 2007

Related U.S. Application Data
(60) Provisional application No. 60/568,481, filed on May 6, 2004.

(51) Int. Cl. A61K 31/405; A61P 1/00

(52) U.S. Cl. 514/415

(57) ABSTRACT

This document involves methods and materials related to increasing the number of interstitial cells of Cajal and treating digestive tract conditions.

### STATEMENT AS TO FEDERALLY SPONSORED RESEARCH

Funding for the work described herein was provided by the federal government, which may have certain rights in the invention.

### BACKGROUND

1. Technical Field

This document relates to methods and materials involved in treating digestive tract conditions. 2. Background Information

Serotonin (5HT) is a neurotransmitter that can interact with many different 5HT receptors. For example, at least 14 different 5HT receptor genes have been identified, forming seven major families of 5HT receptors (5HT1-7). The 5HT3 receptor is a non-selective cation channel, while the other receptors are coupled to heterotrimeric G proteins. While it is increasingly clear that 5HT receptors can couple to more than one G protein, certain generalizations can be made. The 5HT1 family are negatively coupled to adenylyl cyclase and therefore decrease cAMP levels. The 5HT2 family consists of three subtypes including 5HT2B previously called 5HTF because of its high expression in the gastric fundus. 5HT2 receptors are coupled to Gq/11 and phospholipase C. The 5HT4 receptor is coupled to the G protein leading to activation of adenylyl cyclase and increased cAMP levels. 5HT5-7 receptors are less well studied. 5HT7 receptors are positively coupled to adenylyl cyclase via G proteins leading to increased cAMP levels.

### SUMMARY

This document involves methods and materials related to the treatment of digestive tract conditions. For example, this document provides methods and materials that can be used to increase the number of ICC (interstitial cells of Cajal) within a mammal such that a symptom of a digestive tract condition is reduced. ICC are specialized cells of the digestive tract that are involved in normal intestinal motility. The number of ICC can be increased by contacting ICC with 5HT or 5HT receptor agonists (e.g., a 5HT2B receptor agonist). Any digestive tract condition can be treated using 5HT or 5HT receptor agonists to increase the number of ICC within a mammal including, without limitation, irritable bowel syndrome, hypertrophic pyloric stenosis, intestinal pseudoobstruction, gastroparesis (e.g., diabetic gastroparesis and diabetic gastroenteropathy), constipation, Hirschsprung's disease, and functional dyspepsia.

In general, one aspect of this document features a method for increasing the number of interstitial cells of Cajal in a mammal. The method includes (a) identifying a mammal having a reduced number of interstitial cells of Cajal, and (b) administering a serotonin receptor agonist to the mammal under conditions wherein the number of interstitial cells of Cajal in the mammal increases. The mammal can be a human. The step (a) can include identifying the mammal as having the reduced number of interstitial cells of Cajal based on a symptom of a digestive tract disorder. The symptom can result from abnormal digestive tract motility. The symptom can include bloating, nausea, vomiting, constipation, abdominal distention, poor weight gain, or poor growth. The administration can be an oral administration. The serotonin receptor agonist can be a 5HT2B receptor agonist. The serotonin receptor agonist can be serotonin. The serotonin receptor agonist can be m-chlorophenylpiperazine, α-methyl-5-hydroxytryptamine maleate, BW723C86, 1-(2,5-dimethoxy-4-iodophenyl)-2-aminopropane, flumexedol (cerm 1841), quipazine maleate, (+/−)2,5-dimethoxy-4-iodoamphetamine, or 3,4-methylenedioxymethamphetamine. The serotonin receptor agonist can be administered in a time-release capsule. The number of interstitial cells of Cajal in the mammal can increase 5 percent following the administration. The number of interstitial cells of Cajal in the mammal can increase 10 percent following the administration. The number of interstitial cells of Cajal in the mammal can increase 20 percent following the administration. The number of interstitial cells of Cajal in the mammal can increase 30 percent following the administration.

In another embodiment, this document features a method for treating a mammal having a digestive tract condition. The method includes (a) identifying the mammal as having the digestive tract condition, (b) administering a serotonin receptor agonist to the mammal under conditions wherein the number of interstitial cells of Cajal in the mammal increases, thereby reducing a symptom of the digestive tract condition, and (c) observing the reduction of the symptom. The mammal can be a human. The symptom can result from abnormal digestive tract motility. The symptom can be bloating, nausea, vomiting, constipation, abdominal distention, poor weight gain, or poor growth. The administration can be an oral administration. The serotonin receptor agonist can be a 5HT2B receptor agonist. The serotonin receptor agonist can be serotonin. The serotonin receptor agonist can be selected from the group consisting of m-chlorophenylpiperazine, α-methyl-5-hydroxytryptamine maleate, BW723C86, 1-(2,5-dimethoxy-4-iodophenyl)-2-aminopropane, flumexedol (cerm 1841), quipazine maleate, (+/−)2,5-dimethoxy-4-iodoamphetamine, and 3,4-methylenedioxymethamphetamine. The serotonin receptor agonist can be administered in a time-release capsule. The number of interstitial cells of Cajal in the mammal can increase 5 percent following the administration. The number of interstitial cells of Cajal in the mammal can increase 10 percent following the administration. The number of interstitial cells of Cajal in the mammal can increase 20 percent following the administration. The number of interstitial cells of Cajal in the mammal can increase 30 percent following the administration.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a bar graph plotting the number of ICC per high power field (HPF) for cells cultured with or without 5HT. The * indicates that P<0.0004.

FIG. 2 is a bar graph plotting the number of ICC per HPF for control cells or cells treated with 5HT alone or 5HT plus SB204741. The * indicates that P<0.0001 as compared to control cells. The ** indicates that P=<0.003 compared to 5HT treated cells.

FIG. 3 is a photograph of a mouse intramuscular-like interstitial cell of Cajal exhibiting 5HT2B immunoreactivity.

FIG. 4 is a bar graph plotting the normalized copy number of mRNA encoding 5HT2B receptor measured using quantitative real time PCR in control mice and W/W^{V}.

FIG. 5 is a photograph of human jejunum labeled with an antibody against c-Kit (brown) showing myenteric plexus ICC. The arrows indicate where ICC have been captured by Laser Capture Microdissection (LCM).

FIG. 6 is a photograph of a gel containing PCR product produced from RNA extracted from human intestinal cells collected by laser capture microscopy from the ICC myenteric plexus region of the intestine. Bands were detected corresponding to nucleic acid encoding 5HT2B receptors.

FIG. 7 is a photograph of a gel from a single cell reverse transcriptase PCR experiment designed to amplify nucleic acid encoding a 5HT2B receptor. Lanes 1-4 are from single human intestinal interstitial cells of Cajal. Lanes 5 and 6 are from human smooth muscle cells. Lanes 7 and 8 contain 10 μL of bath solution to act as a negative control. The 5HT2B band was present in ICC.

FIG. 8 is a diagram of a chamber for collecting air samples from mice.

FIG. 9 is a graph plotting ¹³CO₂ excretion (mL/min) at the indicated time (minutes) for a control mouse (X) and a mouse with autonomic neuropathy (square).

### DETAILED DESCRIPTION

This document provides methods and materials related to treating digestive tract conditions. For example, this document provides methods and materials that can be used to increase the number of ICC within a mammal such that a symptom of a digestive tract condition is reduced. ICC are specialized cells of the digestive tract that are involved in normal intestinal motility. Abnormalities in ICC can lead to digestive tract disorders. As described herein, the number of ICC can be increased in a mammal by administering 5HT or any other 5HT receptor agonist (e.g., a 5HT2B receptor agonist) to the mammal. Any digestive tract condition can be treated using 5HT or any other 5HT receptor agonist to increase the number of ICC within a mammal including, without limitation, irritable bowel syndrome, hypertrophic pyloric stenosis, intestinal pseudoobstruction, gastroparesis (e.g., diabetic gastroparesis and diabetic gastroenteropathy), constipation, Hirschsprung's disease, and functional dyspepsia. In some cases, the methods and materials provided herein can be used to treat a single digestive tract condition (e.g., a patient with intestinal pseudoobstruction) or a combination of digestive tract conditions (e.g., a patient having both irritable bowel syndrome and constipation).

As described herein, 5HT or any other 5HT receptor agonist can be used to treat a digestive tract condition. The term “5HT receptor agonist” as used herein refers to any compound having the ability to bind to and activate a 5HT receptor including 5HT1, 5HT2, 5HT2B, 5HT3, 5HT4, 5HT5, 5HT6, and 5HT7 receptors. Examples of 5HT receptor agonists that can be used as described herein include, without limitation, serotonin, m-chlorophenyl-piperazine, α-methyl-5-hydroxytryptamine maleate, BW723C86, 1-(2,5-dimethoxy-4-iodophenyl)-2-aminopropane, flumexedol (cerm 1841), quipazine maleate, (+/−)2,5-dimethoxy-4-iodoamphetamine HCL (DOI), and 3,4-methylenedioxymeth-amphetamine. In addition, combinations of different 5HT receptor agonists can be formulated and administered to a mammal. For example, 5HT and m-chlorophenylpiperazine can be combined to form a single capsule for delivery to a mammal.

In general, digestive tract conditions can be treated by contacting digestive tract cells (e.g., small or large intestine cells) with a 5HT receptor agonist. Any method can be used to contact digestive tract cells with a compound such as a 5HT receptor agonist. For example, 5HT receptor agonists can be administered orally so that the administered 5HT receptor agonist contacts digestive tract cells. Other routes of administration that can be used include, without limitation, rectal, intravenous, subcutaneous, and intramuscular administrations.

Before administering a 5HT receptor agonist to a mammal, the mammal can be assessed to determine whether or not the mammal has a digestive tract condition. Any method can be used to determine whether or not a mammal has a digestive tract condition. For example, a mammal (e.g., human) can be identified as having a digestive tract condition upon examination of a tissue biopsy as well as by endoscopic analysis or image analysis techniques (e.g., X rays, CT scans, and magnetic resonance imagery (MRI) scans) or measurements of gut transit and function. In addition, diagnostic methods such as reviewing an individual's prior medical conditions and treatments, interviewing and evaluating an individual, and collecting and analyzing biological samples from an individual can be used to identify the presence of a digestive tract condition. Typically, clinical symptoms or complications can be assessed to determine whether or not a mammal has a digestive tract condition. For example, a mammal can be diagnosed as having irritable bowel syndrome based on the Rome criteria of presence of symptoms (Drossman, Gut, 45 Suppl. 2:II1-5(1999)).

Reviewing an individual's medical history as well as interviewing and evaluating an individual can be helpful in determining the presence of a digestive tract condition since symptoms of digestive tract conditions such as dyspepsia include early satiety, upper abdominal pain, and belching. Collecting and analyzing biological samples from an individual also can help identify a digestive tract condition. In general, biological samples such as stool can be collected and analyzed for signs that indicate digestive tract dysfunction. Such signs can include, without limitation, increased fat levels and abnormal electrolyte levels. Many methods for detecting the presence of these various signs and markers within a biological sample are well known in the art and can be used. For example, the presence of fat within a stool sample can be measured using methods similar to those described elsewhere (Van de Kamer et al., J. Biol. Chem., 177:347-55 (1949)).

In some cases, a tissue biopsy can be collected and analyzed to determine whether or not a mammal has a digestive tract condition. For example, the absence of villi in a small bowel biopsy can indicated that the patient has celiac disease. In some cases, immuno-based assays can be used to detect the presence of one or more signs of a digestive tract condition within a biological sample such as a digestive tract tissue biopsy. Many immuno-based assays are well known in the art including, without limitation, enzyme linked immunosorbent assays (ELISA). Immuno-based assays can use polyclonal antibodies, monoclonal antibodies, or fragments thereof that have high binding affinity for a marker indicative of a digestive tract condition. For example, antibodies against tissue transglutaminase can be used to screen for celiac disease. Such antibodies can be produced using methods described elsewhere (Zeidan et al., Experimental Approaches in Biochemistry and Molecular Biology, William C. Brown Publisher (1996) and Seaver, Commercial Production of Monoclonal Antibodies: A Guide for Scale Up, Marcel Dekker Inc., New York, N.Y. (1987)).

After identifying a mammal as having a digestive tract condition, the mammal can be treated with a 5HT receptor agonist. A 5HT receptor agonist can be administered to a mammal in any amount, at any frequency, and for any duration effective to achieve a desired outcome (e.g., to treat a digestive tract condition). In some cases, a 5HT receptor agonist can be administered to a mammal to increase the number of ICC within a mammal. The increase can be any level of increase including, without limitation, a 5, 10, 25, 50, 75, 100, or more percent increase in the number of ICC within a mammal. For example, the number of ICC detected within a mammal can double following treatment with a 5HT receptor agonist. Any method can be used to determine whether or not the number of ICC have increased within a mammal. For example, the number of ICC within a mammal can be assessed by imaging digestive tract tissue prior to treatment and determining the number of ICC per a fixed surface area or amount of tissue (e.g., 1 cm², 1 cm³, or 10 mg of tissue). After treatment with a 5HT receptor agonist, the number of ICC can be determined again and compared to the number determined prior to treatment.

In some embodiments, a 5HT receptor agonist can be administered to a mammal in an amount, at a frequency, and for a duration effective to increase the number of ICC within a mammal as compared to the number of ICC within a comparable mammal not treated with the 5HT receptor agonist. For example, the number of ICC within a population of mammals (e.g., humans or mice) treated with a 5HT receptor agonist can be compared to the number of ICC within a population of mammals not treated with the 5HT receptor agonist. The increase in ICC number can be any level of increase including, a 5, 10, 25, 50, 75, 100, or more percent increase. For example, the number of ICC can be increased such that the number of ICC detected following treatment with a 5HT receptor agonist doubled. In some cases, a 5HT receptor agonist can be administered to a mammal in an amount, at a frequency, and for a duration effective to increase the number of ICC by more than 100 percent (e.g., 100, 90, 80, 70, 60, 50, 40, 30, 20, 10, or 5 percent) within a one, two, three, four, five, six, seven, eight, nine, ten, eleven, twelve month time period. For example, a 5HT receptor agonist can be administered to double the number of ICC within a mammal within a three, four, five, six, seven, eight, nine, ten or more weeks or within one, two, three, four, five, six, seven, eight, nine, ten, or more months.

An effective amount of a 5HT receptor agonist or formulation containing a 5HT receptor agonist can be any amount that increase the number of ICC within a mammal without producing significant toxicity to the mammal. Typically, an effective amount can be any amount greater than or equal to about 0.5, 1, 10, 20, 30, 40, 50, 60, 70, 80, 90, 100, 125, 150, 175, 200, or more μg/kg (μg per kg of body weight) provided that that amount does not induce significant toxicity to the mammal upon administration. In some cases, the effective amount can be between 1 μg/kg and 3500 μg/kg. If a particular mammal fails to respond to a particular amount, then the amount can be increased by, for example, ten fold. After receiving this higher concentration, the mammal can be monitored for both responsiveness to the treatment and toxicity symptoms, and adjustments made accordingly. For 5HT, an effective amount can be between about 1 μg/kg to about 1 mg/kg of body weight of the mammal per administration when delivered orally. When administered rectally via a suppository or enema, an effective amount can be between 1 μg/kg and 1000 μg/kg. The effective amount can remain constant or can be adjusted as a sliding scale or variable dose depending on the mammal's response to treatment.

To help determine effective amounts of different 5HT receptor agonists, it can be useful to refer to an effective amount equivalent based on the effective amount of a common 5HT receptor agonist. For example, the administration of 5 mg (e.g., twice daily) of 5HT can be an effective amount. The effects produced by this effective amount can be used as a reference point to compare the effects observed for other 5HT receptor agonists used at varying concentrations. Once an equivalent effect is observed, then the specific effective amount for that particular 5HT receptor agonist can be determined. In this case, that particular amount would be termed an 5HT effective amount equivalent.

Various factors can influence the actual effective amount used for a particular application. For example, the frequency of administration, duration of treatment, use of multiple 5HT receptor agonists, route of administration, and severity of the digestive tract condition may require an increase or decrease in the actual effective amount administered.

The frequency of administration can be any frequency that increase the number of ICC within a mammal without producing significant toxicity to the mammal. For example, the frequency of administration can be from about four times a day to about once every other month, or from about once a day to about once a month, or from about one every other day to about once a week. In addition, the frequency of administration can remain constant or can be variable during the duration of treatment. As with the effective amount, various factors can influence the actual frequency of administration used for a particular application. For example, the effective amount, duration of treatment, use of multiple 5HT receptor agonists, route of administration, and severity of the digestive tract condition may require an increase or decrease in administration frequency.

An effective duration of 5HT receptor agonist administration can be any duration that increase the number of ICC within a mammal without producing significant toxicity to the mammal. Thus, the effective duration can vary from several days to several weeks, months, or years. In general, the effective duration for the treatment of a digestive tract condition can range in duration from several days to several months. Once the 5HT receptor agonist administrations are stopped, however, a digestive tract condition may return. Thus, the effective duration for preventing the return of a digestive tract condition can be in some cases for as long as an individual mammal is alive.

Typically, an effective duration can range from about one week to about one, two, three, or more years. Again, prophylactic treatments are typically longer in duration and can last throughout an individual mammal's lifetime.

Multiple factors can influence the actual effective duration used for a particular treatment or prevention regimen. For example, an effective duration can vary with the frequency of 5HT receptor agonist administration, effective 5HT receptor agonist amount, use of multiple 5HT receptor agonists, route of administration, and severity of the digestive tract condition.

A formulation containing a 5HT receptor agonist can be in any form. For example, a formulation containing a 5HT receptor agonist can be in the form of a suppository, enema solution, capsule, pill, tablet, solution, or suspension. In addition, the formulation can contain a cocktail of 5HT receptor agonists. For example, a formulation can contain, without limitation, one, two, three, four, five, or more different 5HT receptor agonists. Further, a formulation containing a 5HT receptor agonist can contain additional ingredients including, without limitation, pharmaceutically acceptable vehicles.

A pharmaceutically acceptable vehicle can be, for example, saline, water, cellulose, starch, and gelatin. Typically, capsules or tablets can contain a 5HT receptor agonist in enteric form. The dose supplied by each capsule or tablet can vary since an effective amount can be reached by administrating either one or multiple capsules or tablets. Any well known pharmaceutically acceptable material can be incorporated into a formulation containing a 5HT receptor agonist including, without limitation, gelatin, cellulose, starch, sugar, or bentonite.

As described herein, any route of administration can be used to administer a 5HT receptor agonist to a mammal. For example, oral or rectal route of administration can be used. In some embodiments, a 5HT receptor agonist can be formulated into a capsule (e.g., time-release capsule, a delayed-release capsule, or a pH-sensitive capsule) designed to deliver 5HT or another 5HT receptor agonist to a particular segment of the digestive tract (e.g., the small intestine or the colon).

After administering a 5HT receptor agonist to a mammal, the mammal can be monitored to determine whether or not a digestive tract condition was treated. For example, a mammal can be assessed after treatment to determine whether or not the number of ICC within the mammal increased. As described herein, any method can be used to assess changes in the number of ICC within a mammal.

The invention will be further described in the following examples, which do not limit the scope of the invention described in the claims.

### EXAMPLES

### Example 1

### Serotonin Increases the Number of ICC

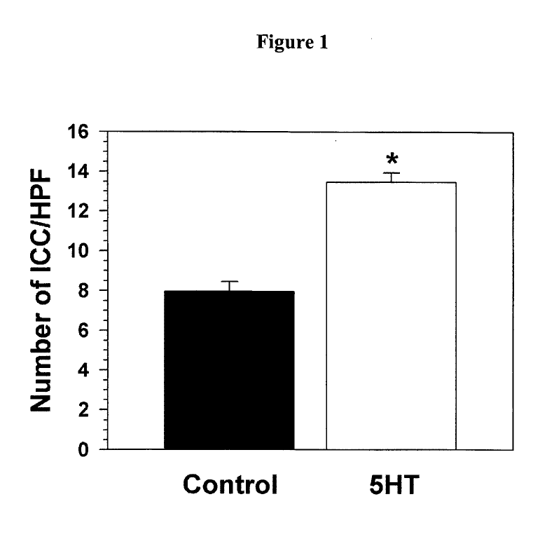

An ICC culture technique was developed and used to test whether 5HT increases ICC number in culture. Mouse intestinal tissue was dissociated, and ICC placed in culture over fibroblasts engineered to make Steel factor (Rich et al., Am. J. Phys. Gastro. Liver Physiol., 284(2):G313-20 (2003)). Blinded parallel cultures were established (n=2, 6 day old Balb/C mice yielding 6 coverslips). 5HT (1 μM) was added to half of the coverslips every 12 hours for 24 hours, after which the cells were fixed. ICC were identified using an antibody to c-kit and counted. Mast cells, which were identified by their characteristic size and round shape, were not included in the analysis. 5HT markedly increased the number of ICC from 8±0.5 ICC per HPF to 14±0.4 ICC per HPF (P<0.0004; FIG. 1). These results demonstrate that 5HT markedly increases the number of ICC.

To determine if the effects of 5HT on increasing the number of ICC were mediated through the 5HT2B receptor, the effect of a 5HT2B receptor antagonist, SB204741, on ICC in culture was tested. Three sets of cultures were set up in parallel with one set being treated with 5HT (1 μM), another set being treated with SB204741 (2 μM) plus 5HT (1 μM), and the last set being untreated controls.

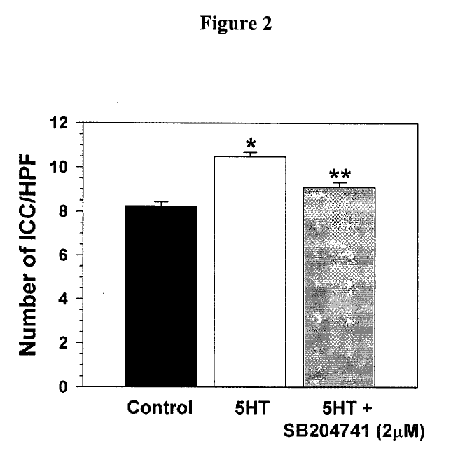

Exposure to 5HT (1 μM) resulted in an increase in ICC number from 8±0.2 ICC per HPF to 11±0.2 ICC per HPF (P<0.0001; FIG. 2). Exposure to SB204741 (2 μM) partly blocked the increase in ICC number induced by serotonin (11±0.2 ICC per HPF to 9±0.2 ICC per HPF; FIG. 2). These results demonstrate that the effect of 5HT on ICC number is mediated via 5HT2B receptors. These results also demonstrate that other 5HT receptors may also be involved because SB204741 did not completely block the increase in ICC number induced by 5HT.

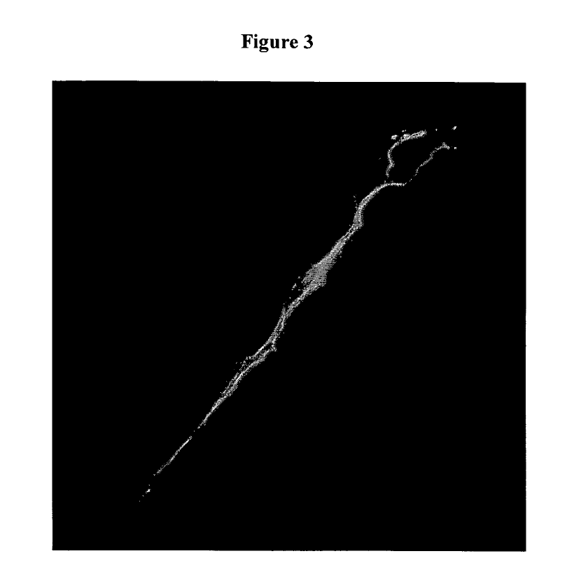

To determine if ICC express 5HT2B receptors, cultured mouse intestine ICC were immunolabeled using an anti-5HT2B antibody. Cells were identified as ICC based on expression of c-Kit as identified by ACK-2. Positive labeling was obtained for 5HT2B receptors (FIG. 3).

Wild-type mice have fundic ICC, while W/W^{V} mice lack fundic ICC. Thus, W/W^{V} mice were used to confirm that fundic ICC express 5HT2B receptors. W/W^{V} mice can be obtained from Jackson labs (strain name: WBB6F1/J-Kit^{W}/Kit^{W-v}).

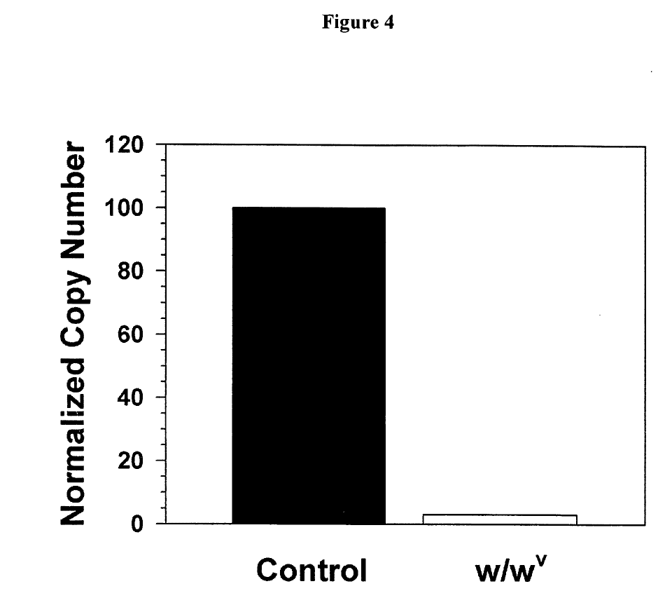

The fundus is the part of the stomach just below the esophagus and is the part that relaxes to accommodate food. Briefly, quantitative real time PCR was used to measure copy number for 5HT2B receptor message in fundi from control mice and W/W^{V} mice. The copy number for 5HT2B mRNA was quantified relative to the calibrator using a standard accepted method (2^{-ΔΔCT}). Message encoding 5HT2B receptor was detected in gastric fundi from control mice to a level substantially greater than the level detected in fundi from W/W^{V} mice lacking fundic ICC (FIG. 4).

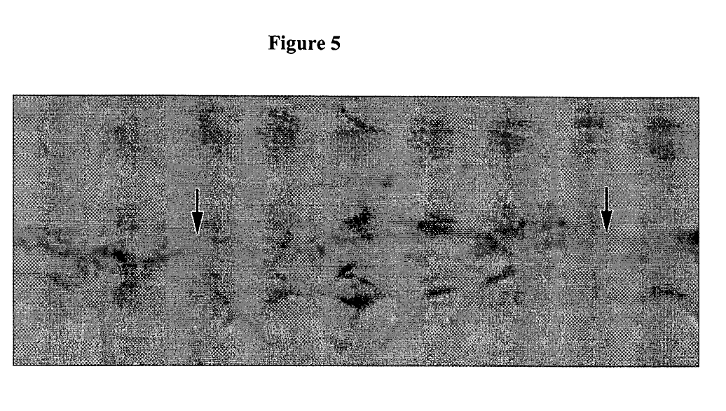

A PixCell® IIe Laser Capture Microdissection System was used to capture ICC from the human jejunum for molecular analysis. This technique allows cells to be captured onto a thermoplastic polymer film coated cap. When the cap is lifted, the selected cells are attached to the film surface while the surrounding tissue remains intact. A 7.5 μm laser spot size was used to precisely collect ICC (FIG. 5).

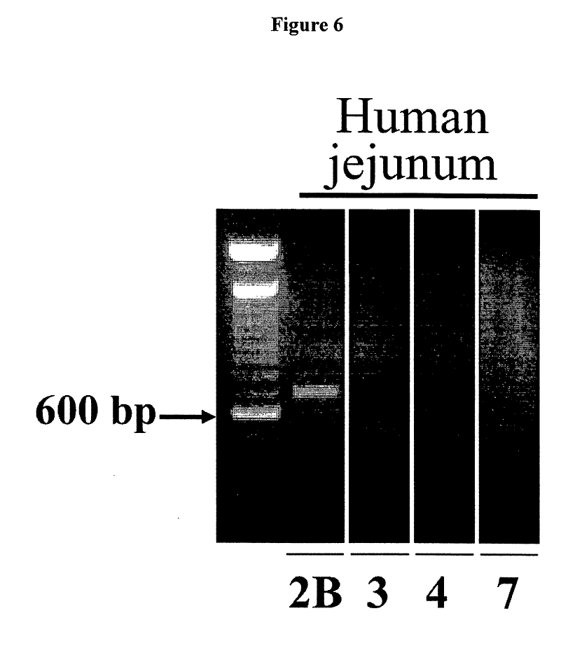

Human myenteric ICC were collected from jejunal tissue sections. About 1000 cells were collected and probed for message RNA encoding 5HT2B, 5HT3, 5HT4, or 5HT7 receptors. 5HT2B message was easily detected in human myenteric ICC (FIG. 6). In addition, a faint band was detected corresponding to nucleic acid encoding 5HT3 receptor.

Single cell RT-PCR was performed to determine whether specific ICC expressed 5HT2B receptors. Briefly, single human intestinal ICC were collected in the tip of a pipette, and the pipette broken into an RT-PCR tube. The pipette's contents were expelled. The RT-PCR tubes containing 0.5 μg of carrier molecule t-RNA and 10 μg proteinase K were immediately frozen on dry ice. Proteinase K rapidly inactivates endogenous nucleases. Incubations at 55° C for 30 minutes followed by 95° C. for 10 minutes were used to destroy the nucleases and proteinase K. Reverse transcription was carried out using GeneAmp Gold RNA PCR Reagent Kit (PE Biosystems, Foster City, Calif.). A mixture of random hexamer and oligo dT primers was used for reverse transcription to obtain fragments of cDNA for subsequent analysis. To obtain enough DNA for sequencing analysis, the DNA product from the first PCR was reamplified through a second PCR. Gene-specific primers were designed to flank exon-exon boundaries. Products amplified from cDNA are therefore smaller than those amplified from genomic DNA, and this size difference was used to detect DNA contamination.

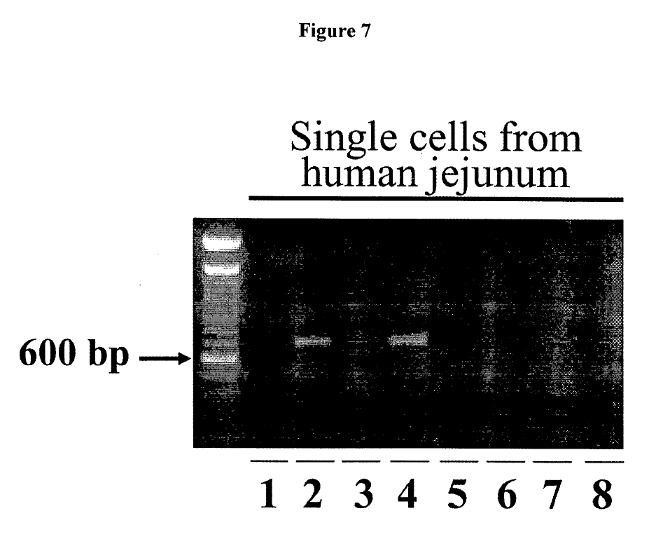

A single cell PCR experiment using primers designed to amplify 5HT2B sequences revealed expression of 5HT2B receptor by ICC and not by smooth muscle cells (FIG. 7).

### Example 2

### Treatment of Digestive Tract Conditions with 5HT and 5HT2B Receptor Agonists

W/W^{V} mice lack certain classes of ICC, and the stomach and small intestine in W/W^{V} mice do not contract well with delayed movement of food through the stomach and intestine. W/W^{V} mice can be used as a model for gastroparesis, intestinal pseudo-obstruction, and constipation.

W/W^{V} mice are sterile. Thus, W mice are bred with W^{V} mice to obtain W/W^{V} mice. Pregnant heterozygote mice are fed a 5HT2B receptor agonist such as m-chlorophenylpiperazine (0.5 mg/kg) or α-methyl-5-hydroxytryptamine maleate (500 nM) starting 2 weeks before birth. The pups are fed the agonist three times a day for an additional 1 to 3 weeks. At 1 to 3 weeks post partum, gastric emptying and the volume of ICC in the stomach and intestine of pups treated with the 5HT2B agonist are compare to the gastric emptying and the volume of ICC in the stomach and intestine observed with control untreated pups.

Gastric emptying is measured as follows. First, the mouse eats octanoic acid (a harmless chemical) mixed with baked egg yolk (0.2 g). The octanoic acid is labeled with stable ¹³C (non radioactive). The octanoic acid is stable in the stomach, but is rapidly absorbed from the small intestine and metabolized. The rate of ¹³CO₂ excretion in the breath closely approximates the gastric emptying rate. The ¹³CO₂ can be determined from breath samples using isotope ratio mass spectrometry, and the level of ¹³CO₂ excretion can be calculated.

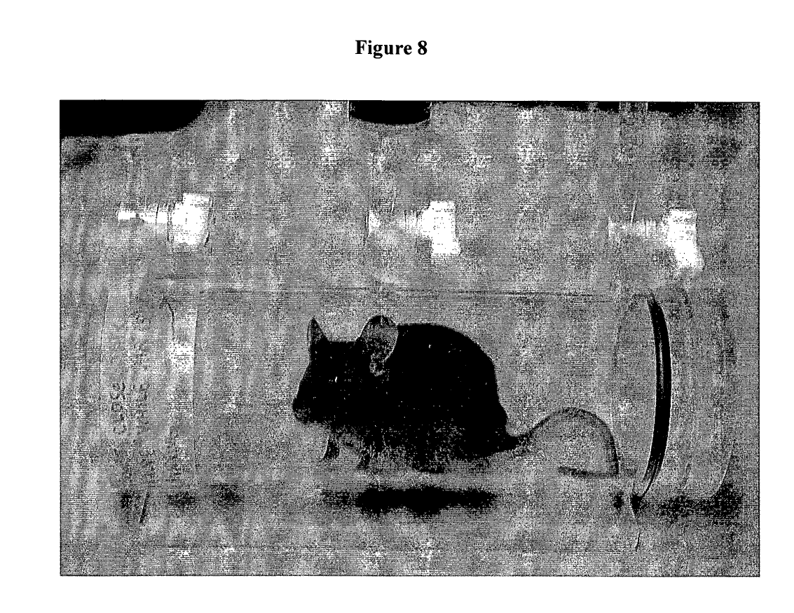

A chamber was built that allows easy placement of the mouse, supplies a constant flow of air through two ports, and has a third port for sampling the chamber (FIG. 8). Samples are obtained every 20 minutes for 3 hours, and ¹³CO₂ is recovered in the breath and measured. A CO₂ production rate of 40 mL/kg/minute is assumed for adult mice. The ¹³CO₂ excretion measurement are analyzed using non-linear regression analysis to fit the curve and calculate gastric half-emptying time.

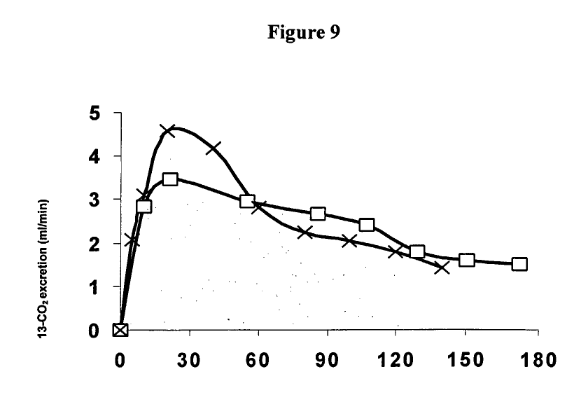

Results from this type of experiment using a control mouse and a mouse with autonomic neuropathy demonstrated that mice with autonomic neuropathy exhibit a detectable delay in gastric emptying (FIG. 9). Thus, this technique allows for the non-invasive measurement of gastric emptying that can be repeated over time.

ICC volume in treated and untreated animals is determined as follows. After the animal is sacrificed, stomach intestine and colon are fixed. The whole mounts are then labeled with an antibody to c-Kit to identify ICC. The whole mount is scanned at 0.6 μm intervals to reconstruct the entire thickness of the wall. The images are then imported into a software program called Analyze developed at the Mayo Clinic, and the volume of c-Kit positive cells measured. Mast cells are also c-Kit positive, but are round and are excluded from analysis.

In a separate experiment, a 5HT2B receptor agonist is given to adult Balb/c mice (n=6 for each drug) for 2 weeks. Gastric emptying is measured both pre- and post-agonist treatment. In addition, the mice are sacrificed, and ICC volumes calculated and compared to those obtained with control mice.

In another experiment, female NOD/LtJ 001976 mice, which can be used as a model for gastroparesis, are obtained from Jackson labs and allowed to become diabetic as indicated by a non-fasting plasma glucose level higher than 250 mg/dL. Four weeks after becoming diabetic, gastric emptying is measured. The mice are then fed a 5HT2B agonist (e.g., m-chlorophenylpiperazine, 0.5 mg/kg, or α-methyl-5-hydroxytryptamine maleate, 500 nM) for a period of time (e.g., about 2 weeks). At that point, the mice are retested for gastric emptying. ICC volumes are also measured as described above in diabetic NOD mice controls and diabetic NOD mice fed a 5HT2B agonist.

To determine the effect of loss of ICC in the intestine, a mouse model of partial small bowel obstruction is used to induce loss of ICC and determine if ICC loss is prevented or recovered faster by use of a 5HT2B agonist. In this model, a ring is placed around the distal small bowel for up to 10-14 days, resulting in smooth muscle hypertrophy and loss of ICC for a distance of up to 100 mm (in a graded fashion) orad to the ring. A non-obstructive polyethylene ring that is one mm wider than the outside diameter of the small intestine with a slit on one end is used. To further reduce mortality, the mice are maintained on a liquid diet of Ensure® diluted with an equal volume of water beginning 12 hours before surgery.

Mice were maintained on this diet for 3 weeks with normal weight gain and no apparent ill health. Using these modifications, the mortality rate of the mice, including death during surgery, can be reduced to 20%. Marked smooth muscle hypertrophy was observed by day 4 and loss of c-Kit positive cells was observed after day 5.

Mice are fed a 5HT2B agonist starting 1 day before the surgery and are sacrificed at day 10. The ICC loss in mice fed a 5HT2B agonist is compared to the ICC loss observed in control mice not fed the agonist.

### Example 3

### Serotonin Increases the Number of ICC from Adults

To determine the effects 5HT has on cells from adult mice, an organotypic culture was used. Briefly, one cm segments of mouse small intestine were cut, pinned flat mucosa side up, stripped of mucosa and submucosa layers, and placed into fresh, sterile-filtered Kreb's solution with 3 mM antibiotic. Krebs was removed, and DMEM (Dulbecco's Modified Eagle's Media) with 10% FBS (Fetal Bovine Serum) and 1% antibiotic/antimycotic was added to the culture. Explants from 6 month old mice were incubated at 37° C., 5% CO₂.5HT (1 μM) was added in fresh medium twice daily. In the control explant, medium was added without 5HT. The tissue was fixed at day 8 and stained using an antibody to c-Kit. Images were collected using a confocal microscope and reconstructed in 3D, and the volume of ICC calculated. The stack of confocal images revealed the preservation of ICC networks in the organotypic culture at day 8 and the greater number of ICC in the explant exposed to 5HT. These results indicate that 5HT can also modulate ICC numbers in adult mice.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for increasing the number of interstitial cells of Cajal in a mammal, said method comprising:
(a) identifying a mammal having a reduced number of interstitial cells of Cajal, and (b) administering a serotonin receptor agonist to said mammal under conditions wherein the number of interstitial cells of Cajal in said mammal increases.

2. The method of claim 1, wherein said mammal is a human.

3. The method of claim 1, wherein said step (a) comprises identifying said mammal as having said reduced number of interstitial cells of Cajal based on a symptom of a digestive tract disorder.

4. The method of claim 3, wherein said symptom results from abnormal digestive tract motility.

5. The method of claim 3, wherein said symptom is selected from the group consisting of bloating, nausea, vomiting, constipation, abdominal distention, poor weight gain, and poor growth.

6. The method of claim 1, wherein said administration is an oral administration.

7. The method of claim 1, wherein said serotonin receptor agonist is a 5HT2B receptor agonist.

8. The method of claim 1, wherein said serotonin receptor agonist is serotonin.

9. The method of claim 1, wherein said serotonin receptor agonist is selected from the group consisting of m-chlorophenylpiperazine, α-methyl-5-hydroxytryptamine maleate, BW723C86, 1-(2,5-dimethoxy-4-iodophenyl)-2-aminopropane, flumexedol (cerm 1841), quipazine maleate, (+/−)2,5-dimethoxy-4-iodoamphetamine, and 3,4-methylenedioxymethamphetamine.

10. The method of claim 1, wherein said serotonin receptor agonist is administered in a time-release capsule.

11. The method of claim 1, wherein the number of interstitial cells of Cajal in said mammal increases 5 percent following said administration.

12. The method of claim 1, wherein the number of interstitial cells of Cajal in said mammal increases 10 percent following said administration.

13. The method of claim 1, wherein the number of interstitial cells of Cajal in said mammal increases 20 percent following said administration.

14. The method of claim 1, wherein the number of interstitial cells of Cajal in said mammal increases 30 percent following said administration.

15. A method for treating a mammal having a digestive tract condition, said method comprising:
(a) identifying said mammal as having said digestive tract condition,
(b) administering a serotonin receptor agonist to said mammal under conditions wherein the number of interstitial cells of Cajal in said mammal increases, thereby reducing a symptom of said digestive tract condition, and
(c) observing the reduction of said symptom.

16. The method of claim 15, wherein said mammal is a human.

17. The method of claim 15, wherein said symptom results from abnormal digestive tract motility.

18. The method of claim 15, wherein said symptom is selected from the group consisting of bloating, nausea, vomiting, constipation, abdominal distention, poor weight gain, and poor growth.

19. The method of claim 15, wherein said administration is an oral administration.

20. The method of claim 15, wherein said serotonin receptor agonist is a 5HT2B receptor agonist.

21. The method of claim 15, wherein said serotonin receptor agonist is serotonin.

22. The method of claim 15, wherein said serotonin receptor agonist is selected from the group consisting of m-chlorophenylpiperazine, α-methyl-5-hydroxytryptamine maleate, BW723C86, 1-(2,5-dimethoxy-4-iodophenyl)-2-aminopropane, flumexedol (cerm 1841), quipazine maleate, (+/−)2,5-dimethoxy-4-iodoamphetamine, and 3,4-methylenedioxymethamphetamine.

23. The method of claim 15, wherein said serotonin receptor agonist is administered in a time-release capsule.

24. The method of claim 15, wherein the number of interstitial cells of Cajal in said mammal increases 5 percent following said administration.

25. The method of claim 15, wherein the number of interstitial cells of Cajal in said mammal increases 10 percent following said administration.

26. The method of claim 15, wherein the number of interstitial cells of Cajal in said mammal increases 20 percent following said administration.

27. The method of claim 15, wherein the number of interstitial cells of Cajal in said mammal increases 30 percent following said administration.
